(19) United States
(12) Patent Application Publication
(10) Pub. No.: US 2012/0270830 A1
(43) Pub. Date: Oct. 25, 2012

(54) NUTRITIONAL COMPOSITIONS INCLUDING THEANINE AND EXOGENOUS NUCLEOTIDES

(75) Inventors: Norman Alan Greenberg, New Hope, MN (US); Peter Geoffrey McLean, Lausanne (CH); Magali Faure, Forel (CH)

(73) Assignee: NESTEC S.A., Vevey (CH)

(21) Appl. No.: 13/497,403

(22) PCT Filed: Aug. 23, 2010
(86) PCT No.: PCT/US10/46301

§ 371 Date: Apr. 30, 2012

Related U.S. Application Data
(60) Provisional application No. 61/245,827, filed on Sep. 25, 2009.
(60) Provisional application No. 61/263,454, filed on Nov. 23, 2009.

(51) Int. Cl. A61K 31/7052; A61P 1/00; A61K 31/7068; A61K 31/7072; A61K 31/7076; A61K 31/708

(52) U.S. Cl. 514/47; 514/43; 514/48; 514/49; 514/51

(57) ABSTRACT

Nutritional compositions and methods of making and using the nutritional compositions are provided. In a general embodiment, the present disclosure provides a nutritional composition including theanine and one or more exogenous nucleotides. The nutritional compositions can be specifically used to treat functional bowel disorders, including inflammatory bowel syndrome.

### BACKGROUND

The present disclosure generally relates to health and nutrition. More specifically, the present disclosure relates to nutritional compositions including theanine and an exogenous nucleotide and methods of making and using the nutritional compositions.

There are many types of nutritional compositions currently on the market. Nutritional compositions can be targeted toward certain consumer types, for example, young, elderly, athletic, etc., based on the specific ingredients of the nutritional composition. Nutritional compositions can also be formulated based on the certain physiological conditions that the nutritional compositions are intended to treat or improve.

Certain widespread physiological conditions include irritable bowel syndrome (“IBS”) and other functional hovel disorders. IBS and other functional bowel disorders conditions can be characterized by abdominal pain, cramping, bloating, diarrhea and/or constipation. IBS and other functional bowel disorders cause a great deal of discomfort and distress, but it does not permanently harm the intestines and does not lead to a serious disease. For some people, IBS and other functional bowel disorders can be disabling b preventing them from working, attending social events, or even traveling short distances. As much as 20 percent of the adult population, or one in five Americans, have symptoms of MS and other functional bowel disorders, making it one of the most common disorders diagnosed by doctors.

### SUMMARY

Nutritional compositions having theanine and an exogenous nucleotide and methods of making and using the nutritional compositions are provided. In a general embodiment, the present disclosure provides a nutritional composition including theanine and one or more exogenous nucleotides. The nutritional composition can be a complete feeding or as an oral nutritional supplement.

The nutritional composition can be in a formulation designed for any mammal such as a human or an animal. The active ingredients in the nutritional composition can also be provided as a modular product. A modular product can be defined as a method of delivering one or more specific nutrients as a supplement and not intended to be used for sole source nutrition. In addition, the nutritional compositions can be shelf stable and exhibit good shelf life at ambient or even above ambient temperatures that may be encountered during distribution.

The exogenous nucleotide can be in a monomeric form such as for example, 5′ Adenosine Monophosphate (“5′-AMP”), 5′-Guanosine Monophosphate (“5′-GMP”), 5-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”) or a combination thereof. The exogenous nucleotide can also be in a polymeric form such as, for example, an intact ribonucleic acid.

In an embodiment, the nutritional composition further includes one or more prebiotics. The prebiotic can be acacia gum, alpha glucan, beta glucan, fructooligosaccharides, galactooligosaccharides, gentiooligosaccharides, glucooligosaccharides, guar gum, inulin, isomaltooligosaccharides, lactosucrose, lactulose, partially hydrolyzed guar gum, pecticoligosaccharides, soyoligosaccharides, sugar alcohols, xylooligosaccharides, or a combination thereof or a combination thereof.

In an embodiment, the nutritional composition further includes one or more probiotics. The probiotic can be Saccharomyres, Debaromyces, Candida, Pichia, Torulopsis, Aspergillus, Rhizopus, Mucor, Penicillium, Bifidobacteritun, Bacteroides, Clostridium, Fusobacterium, Milissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus or a combination thereof.

In another embodiment, the nutritional composition further includes one or more amino acids. The ammo acid can be Alanine, Arginine, Asparagine, Aspartate, Cysteine, Glutamate, Glutamine, Glycine, Histidine, Isoleucine, Leucine, Lysine, Methionine, Phenylalanine, Proline, Serine, Threonine, Tryptophan, Tyrosine, and Valine or a combination thereof.

In an embodiment the nutritional composition further includes one or more synbiotics, fish oils, phytonutrients, antioxidants, and/or partially hydrolyzed guar gum.

In an embodiment, the nutritional composition is in an administerable form such as pharmaceutical formulations, nutritional formulations, dietary supplements, functional foods, beverage products or a combination thereof.

In another embodiment, the present disclosure provides a method of making a nutritional composition. The method comprises adding theanine and an exogenous nucleotide to a nutritional composition.

In an alternative embodiment the present disclosure provides a method of treating irritable bowel syndrome in an individual or mammal. The method comprises administering to an individual or mammal in need of same a nutritional composition including an effective amount of theanine and one or more exogenous nucleotides.

An advantage of the present disclosure is to provide an improved nutritional composition having theanine and exogenous nucleotides.

Another advantage of the present disclosure is to provide a method of making an improved nutritional composition.

Yet another advantage of the present disclosure is to provide a nutritional composition that treats irritable bowel syndrome.

Additional features and advantages are described herein, and will be apparent from the following Detailed Description.

### DETAILED DESCRIPTION

The present disclosure relates to nutritional compositions including theanine and exogenous nucleotides and methods of making and using the nutritional compositions. Embodiments of the nutritional compositions of the present disclosure can take advantage of the gut brain axis and allow for theanine to reduce the perceived stress and anxiety level of IBS and other functional bowel disorder patients. Theanine in combination with nucleotides can improve blood flow and provide preformed substrates for rapidly dividing cells that improve the functionality of the gastrointestinal tract; e.g., barrier, digestive enzyme levels, and immunity thereby reducing functional bowel disorder symptoms. Optional ingredients such as partially hydrolyzed guar gum may further improve IBS and other functional bowel disorder symptoms.

In a general embodiment, the present disclosure provides a nutritional composition including theanine and one or more exogenous nucleotides. The combination of these two ingredients can provide a synergistic benefit achieved by targeting the brain-gut axis that can further improve the benefits demonstrated by nucleotides in IBS and other functional bowel disorder patients.

Theanine (gamma-glutamylethylamide, or 5-N-ethyl-glutamine) is a glutamic acid analog or amino acid derivative commonly found in tea (infusions of Camellia sinensis), and also in the basidiomycete mushroom Boletus badius. The exogenous nucleotides can be in the form of monomers and polymers as part of the nutritional compositions, for example, that serve to improve the cell energy charge and thus maintain the ability of the cell to maintain anabolic processes including protein synthesis.

A nucleotide is a subunit of deoxyribonucleic acid (“DNA”) or ribonucleic acid (“RNA”). It is an organic compound made up of a nitrogenous base, a phosphate molecule, and a sugar molecule (deoxyribose in DNA and ribose in RNA). Individual nucleotide monomers (single units) are linked together to form polymers, or long chains. The exogenous nucleotides in embodiments of the present disclosure are specifically provided by dietary supplementation.

The exogenous nucleotides can be in a monomeric form such as, for example, 5′ Adenosine Monophosphate (“5′-AMP”), 5′-Guanosine Monophosphate (“5-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”) or a combination thereof. The exogenous nucleotides can also be in a polymeric form such as, for example, an intact RNA. There can be multiple sources of the polymeric form such as, for example, yeast RNA.

As used herein, the term “nutritional composition” includes, but is not limited to, complete nutritional compositions, partial or incomplete nutritional compositions, and disease or condition specific nutritional compositions. A complete nutritional composition (i.e. those which contain all the essential macro and micro nutrients) can be used as a sole source of nutrition for the patient. Patients can receive 100% of their nutritional requirements from such complete nutritional composition. A partial or incomplete nutritional composition does not contain all the essential macro and micro nutrients and cannot be used as a sole source of nutrition for the patient. Partial or incomplete nutritional compositions can be used as a nutritional supplement. A disease or condition specific nutritional composition is a composition that delivers nutrients or pharmaceuticals and can be a complete or partial nutritional composition.

As used herein, “about,” is preferably understood to refer to numbers in a range of numerals. Moreover, all numerical ranges herein should be understood to include all integer, whole or fractions, within the range.

As used herein, animals include, but is not limited to mammals which includes but is not limited to rodents, aquatic mammals, domestic animals such as dogs and cats, farm animals such as sheep, pigs, cows and horses, and humans. Wherein the terms animal or mammal or their plurals are used, it is contemplated that it also applies to any animals that are capable of the effect exhibited or intended to be exhibited by the context of the passage.

As used herein the term “antioxidant” is preferably understood to include any one or more of various substances (as beta-carotene (a vitamin A precursor), vitamin C, vitamin E, and selenium) that inhibit oxidation or reactions promoted by Reactive Oxygen Species (ROS) and other radical and non-radical species. Additionally, antioxidants are molecules capable of slowing or preventing the oxidation of other molecules. As used herein, non-limiting examples of antioxidants include carotenoids, coenzyme Q10 (“CoQ10”), flavonoids, glutathione Goji (Wolfberry), hesperidine, Lactowolfberry, lignan, lutein, lycopene, polyphenols, selenium, vitamin A, vitamin B1, vitamin B6, vitamin B12, vitamin C, vitamin D, vitamin E, and combinations thereof

As used herein, “effective amount” is preferably an amount that prevents a deficiency, treats a disease or medical condition in an individual or more generally, reduces symptoms, manages progression of the diseases or provides a nutritional, physiological, or medical benefit to the individual. A treatment can be patient- or doctor-related. In addition, while the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, “Food grade micro-organisms” means micro-organisms that are used and generally regarded as safe for use in food.

As used herein, “Long term administrations” are preferably continuous administrations for more than 6 weeks.

As used herein, mammal—includes but is not limited to rodents, aquatic mammals, domestic animals such as dogs and cats, farm animals such as sheep, pigs, cows and horses, and humans. Wherein the term mammal is used, it is contemplated that it also applies to other animals that are capable of the effect exhibited or intended to be exhibited by the mammal.

The term “microorganism” is meant to include the bacterium, yeast and/or fungi, a cell growth medium with the microorganism or a cell growth medium in which microorganism was cultivated.

As used herein, a “nucleotide” is preferably understood to be a subunit of deoxyribonucleic acid (“DNA”) or ribonucleic acid (“RNA”). It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation. The exogenous nucleotide can be in a monomeric form such as, for example, 5′ Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”) or a combination thereof. The exogenous nucleotide can also be in a polymeric form such as, for example, an intact RNA. There can be multiple sources of the polymeric form such as, for example, yeast RNA.

Nutritional products is preferably understood to further include any number of additional ingredients, including, for example one or more, vitamin, mineral, sugar, a pharmaceutically acceptable carrier, excipient, flavor agent, or colorants.

As used herein, “phytochemicals” or “phytonutrients” are non-nutritive compounds that are found in many foods. Phytochemicals are functional foods that have health benefits beyond basic, nutrition, and are health promoting compounds that come from plant sources. As used herein, “Phytochemicals” and“Phytonutrients” refers to any chemical produced by a plant that imparts one or more health benefit on the user. Phytochemicals can be administered by any means, including topically, enterally, and/or parenterally. As used herein, non-limiting examples of phytochemicals and phytonutrients include those that are:
1. Phenolic compounds which include Monophenols (such as Apiole, Carnosol, Carvacrol, Dillapiole, Rosemarinol); Flavonoids (polyphenols) including Flavonols (such as: Quercetin, Gingerol, Kaempferol, Myricetin, Rutin, Isorhamnetin), Flavanones (such as: Hesperidin, Naringenin, Silybin, Eriodictyol), Flavones (such as: Apigenin, Tangeritin, Luteolin), Flavan-3-ols (such as: Catechins, (+)-Catechin, (+)-Gallocatechin, (−)-Epicatechin, (−)-Epigallocatechin, (−)-Epigallocatechin gallate (EGCG), (−)-Epicatechin 3-gallate, Theaflavin, Theaflavin-3-gallate, Theaflavin-3′-gallate, Theaflavin-3,3′-digallate, Thearubigins), Anthocyanins (flavonals) and Anthocyanidins (such as: Pelargonidin, Peonidin, Cyanidin, Delphinidin, Malvidin, Petunidin), Isoflavones (phytoestrogens) (such as: Daidzein (formononetin), Genistein (biochanin A), Glycitein), Dihydroflavonols, Chalcones, Coumestans (phytoestrogens), and Coumestrol; Phenolic acids (such as: Ellagic acid, Gallic acid, Tannic acid, Vanillin, Curcumin); Hydroxycinnamic acids (such as: Caffeic acid, Chlorogenic acid, Cinniamic acid, Ferulic acid, Coumarin); Lignans (phytoestrogens), Silymarin, Secoisolariciresinol, Pinoresinol and lariciresinol); Tyrosol esters (such as: Tyrosol, Hydroxytyrosol, Oleocanthal, Oleuropein); Stilbenoids (such as: Resveratrol, Pterostilbene, Picetannol) and Punicalagins;
2. Terpenes (isoprenoids) which include Carotenoids (tetraterpenoids) including Carotenes (such as: α-Carotene, β-Carotene, γ-Carotene, δ-Carotene, Lycopene, Neurosporene, Phytofluene, Phytoene), and Xanthophylls (such as Canthaxanthin, Cryptoxanthin, Zeaxanthin, Astaxanthin, Lutein, Rubixanthin); Monoterpenes (such as: Limonene, Perillyl alcohol); Saponins; Lipids including: Phytosterols (such as: Campesterol, beta Sitosterol, gamma sitosterol, Stigmasterol), Tocopherols (vitamin E), and ornega-3, 6, and 9 fatty acids (such as: gamma-linolenic acid); Triterpenoid (such as: Oleanolic acid, Ursolic acid, Betulinic acid, Moronic acid);
3. Betalains which include Betacyanins (such as: betanin, isobetanin, probetanin, neobetanin); and Betaxanthins (non glycosidic versions) (such as: Indicaxanthin, and Vulgaxanthin);
4 Organosulfides which include Dithiolthiones (isothiocyanates) (such as: Sulphoraphane); and Thiosulphonates (allium compounds) (such as: Allyl methyl trisulfide, and Diallyl sulfide), Indoles, glucosinolates which include Indole-3-carbinol; sulforaphane; 3,3′-Diindolylmethane; Sinigrin; Allicin; Alliin; Allyl isothiocyanate; Piperine; Syn-propanethial-S-oxide;
5. Protein inhibitors which include protease inhibitors,
6. Other organic acids which include Oxalic acid, Phytic acid (inositol hexaphosphate); Tartaric acid; and Anacardic acid; and
7. combinations thereof.

As used in this specification and the appended claims, the singular forms “a”, “an” and “the” include plural referents unless the context clearly dictates otherwise. Thus, for example, reference to “a polypeptide” includes a mixture of two or more polypeptides, and the like.

As used herein, a “Prebiotic” is preferably a food substances that selectively promote the growth of beneficial bacteria or inhibit the growth of pathogenic bacteria in the intestines. They are not inactivated in the stomach and/or upper intestine or absorbed in the GI tract of the person ingesting them, but they are fermented by the gastrointestinal microflora and/or by probiotics. Prebiotics are for example defined by Glenn R. Gibson and Marcel B. Roberfroid, Dietary Modulation of the Human Colonic Microbiota: Introducing the Concept of Prebiotics. 3. Nutr. 1995 125: 1401-1412.

As used herein, Probiotics micro-organisms (hereinafter “probiotics”) are preferably microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or wellbeing of the host. (Salminen S, Ouwehand A. Benno Y. et al “Probiotics: how should they be defined” Trends Food Sci. Technol. 1999:10 107-10). In general, it is believed that these micro-orgariisms inhibit or influence the growth and/or metabolism of pathogenic bacteria in the intestinal tract. The probiotics may also activate the immune function of the host. For this reason, there have been many different approaches to include probiotics into food products.

The term protein “oligopeptides” or “polypeptide” as used herein is preferably understood to refer to any composition that includes, a single amino acids (monomers), two or more amino acids joined together by a peptide bond (dipeptide, tripeptide, or polypeptide), precursor, homolog, analog, mimetic, salt, prodrug, metabolite, or fragment thereof or combination. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-caboxylation, glycation, glycosylation, glycosylphosphatidyl inositol (GPI) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide

All dosage ranges contained within this application are intended to include all numbers, whole or fractions, contained within said range.

As used herein, “Short term administrations” are preferably continuous administrations for less than 6 weeks.

As used herein, a synbiotic is a supplement that contains both a prebiotic and a probiotic that work together to improve the microflora of the intestine.

As used herein, the terms “treatment”, “treat” and “to alleviate” is preferably to both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” also refer to the maintenance and/or promotion of health in an individual not suffering from a disease but who may be susceptible to the development of an unhealthy condition, such as nitrogen imbalance or muscle loss. The terms “treatment”, “treat” and “to alleviate” are also intended to include the potentiation or otherwise enhancement of one or more primary prophylactic or therapeutic measure.

As used herein, a “tube feed” is preferably a complete or incomplete nutritional products that are administered to an animal's gastrointestinal system, other than through oral administration, including but not limited to a nasogastric tube, orogastric tube, gastric tube, jejunostomy tube (J-tube), percutaneous endoscopic gastrostomy (PEG), port, such as a chest wall port that provides access to the stomach, jejunum and other suitable access ports.

As used herein the term “vitamin” is preferably understood to include any of various fat-soluble or water-soluble organic substances (non-limiting examples include vitamin A, vitamin B1, vitamin B6, vitamin B12, vitamin C, vitamin D, vitamin E) essential in minute amounts for normal growth and activity of the body and obtained naturally from plant and animal foods or synthetically made, pro-vitamins, derivatives,. analogs.

As used herein, the term “functional bowel disorders” includes:
a. Dyspepsia—A symptom or set of symptoms that are considered by most physicians to originate from the gastroduodenal region. The specific symptoms are postprandial fullness, early satiation, bloating in the upper abdomen, epigastric pain or epigastric burning, nausea, vomiting, and belching. One or more symptoms are required for the diaziosis.
b. Irritable bowel syndrome—A disorder in which abdominal pain or discomfort is associated with defecation or a change in bowel habit, and with features of disordered defecation.
c. Functional abdominal pain syndrome—Also called “chronic idiopathic abdominal pain” and “chronic functional abdominal pain” describes pain attributed to the abdomen that is poorly related to gut function, is associated with some loss of daily activities, and has been present for at least 6 months. The pain is constant, nearly constant, or at least frequently recurring.
d. Functional anorectal disorders—subdivided into
   Functional fecal incontinence—Recurrent uncontrolled passage of fecal material for at least 3 months. There is also involuntary passage of flatus.
   ii. Functional anorectal pain—Two forms chronic proctalgia and proctalgia fugax.
      1. Chronic proctalgia—also called levator ani syndrome, levator spasm, puborectalis syndrome, pyriformis syndrome, and pelvic tension myalgia. The pain is vague, dull ache or pressure sensation high in the rectum.
      2. Proctalgia fugax—Sudden severe pain in the anal area lasting several seconds to as long as 30 minutes, and then disappearing completely. Pain is mostly (90% of cases) localized and infrequent.
e. Functional defecation disorders—Slow colonic transit or outlet delay. They are characterized by paradoxical contraction or inadequate relaxation of the pelvic floor muscles or inadequate impulsive forces during attempted defecation. They are associated with excessive straining, feeling of incomplete evacuation, and digital facilitation of bowel movements.

The exogenous nucleotides can be combined with other ingredients for the repair and maintenance of function and structure. For example, the exogenous nucleotides could work more effectively to support the repair, maintenance, and improvement of skeletal muscle when used in combination with other specific food components such as amino acids (e.g., leueine), lipids to modulate inflammation (low n6:n3), bioactive peptides, protease inhibitors, creatine, etc.

In an embodiment, the nutritional composition further includes one or more prebiotics. Non-limiting examples of prebiotics include fructooligosaccharides, inulin, lactulose, galactooligosaccharides, acacia gum, soyoligosaccharides, xylooligosaccharides, isomaltooligosaccharides, gentiooligosaccharides, lactosucrose, glucooligosaccharides, pecticoligosaccharides, resistant starches, sugar alcohols or a combination thereof.

In an embodiment, the nutritional composition further includes one or more probiotics. As used herein, probiotics are defined as microorganisms (e.g., live, reduce capacity to grow and replicate, or non-replicating) that could confer health benefits on the host when administered in adequate amounts. Non-limiting examples of probiotics include acacia gum, alpha glucan, beta glucan, fructooligosaccharides, galactooligosaccharides, gentiooligosaccharides, glucooligosaccharides, guar gum, inulin, isomaltooligosaccharides, lactosucrose, lactulose, partially hydrolyzed guar gum, pecticoligosaccharides, soyoligosaccharides, sugar alcohols, xylooligosaccharides, or a combination thereof.

In another embodiment, the nutritional composition further includes one or more amino acids. Non-limiting examples of amino acids include Alanine, Arginine, Asparagine, Aspartate, Cysteine, Glutamate, Glutamine, Glutamine, Glycine, Histidine, Isoleucine, Leucine, Lysine, Methionine, Phenylalanine, Proline, Serine, Threonine, Tryptophan, Tyrosine, and Valine or a combination thereof. Some amino acids have a beneficial effect of the gastrointestinal tract, for example. Threonine has a role in mucin production, additionally glutamine, glycine, and cysteine has a role in glutathione production.

In an embodiment, the nutritional composition further includes one or more symbiotics, fish oils, phytonutrients and/or antioxidants. As used herein, a synbiotic is a supplement that contains both a prebiotic and a probiotic that work together to improve the microflora of the intestine. Non-limiting examples of fish oils include docosahexaenoic acid (“DHA”) and eicosapentaenoic acid (“EPA”). Docosahexaenoic acid (“DHA”) and or eicosapentaenoic acid (“EPA”) may also be present from a non-Fish Oil source. Non-limiting examples of phytonutrients non-limiting examples of phytonutrients include those that are flavonoids and allied phenolic and polyphenolic compounds, terpenoids such as carotenoids, and alkaloids; including curcumin, limonin, and quercetin.

In another embodiment, the present disclosure provides a method of making a nutritional composition. The method comprises adding theanine and one or more exogenous nucleotides to a nutritional composition in an effective amount, for example, to treat a diseased condition or improve the health of the mammal. The nutritional composition can be in an administerable form such as pharmaceutical formulations, nutritional formulations, dietary supplements, functional foods, beverage products or a combination thereof.

In an alternative embodiment, the present disclosure provides a method of treating irritable bowel syndrome in an individual (e.g., human, patient) or a mammal (e.g., pet, animal). The method comprises administering to an individual or mammal having irritable bowel syndrome a nutritional composition including an effective amount of theanine and one or more exogenous nucleotides. For example, the nutritional composition can include the theanine in an amount to be administered ranging from about 100 mg/day to about 600 mg/day, preferably 100 mg/day to about 400 mg/day, preferably 150 mg/day to about 400 mg/day, preferably 200 mg/day to about 300 mg/day.

The nutritional composition can include the exogenous nucleotides m an amount to be administered ranging from about 200 mg/day to about 6 grams/day, preferably 500 mg/day to 4 grams/day, preferably 750 mg/day to 3.5 grams/day, or preferably 1 gram/day to 3 grams/day.

Other optional ingredients can be added to make the nutritional composition sufficiently palatable. The optional ingredients can be added in any suitable amount. For example, the optional ingredient partially hydrolyzed guar gum can be administered at a level of about 3 grams/day to about 15 grams/day, preferably 4 grams/day to 12 grams/day, preferably of 5 grams/day-10 grams/day.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A nutritional composition comprising theanine and an endogenous nucleotide.

2. The nutritional composition of claim 1 comprising an effective amount of theanine and an effective amount of exogenous nucleotide for treating a Functional Bowel Disorder in a mammal.

3. The nutritional composition of claim 1, wherein the exogenous nucleotide is in a monomeric form selected from the group consisting of 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate and combinations thereof.

4. The nutritional composition of claim 1, wherein the exogenous nucleotide is intact ribonucleic acid.

5. The nutritional composition of claim 1 comprising at least one ingredient selected from the group consisting of prebiotic, prebiotic, symbiotic, amino acid, phytonutrients, antioxidant, and combinations thereof.

6. The nutritional composition of claim 1 comprising partially hydrolyzed guar gum.

7. The nutritional composition of claim 6, wherein the nutritional composition can be administered and provide partially hydrolyzed guar gum in an amount ranging from about 3 g/day to about 15 grams/day.

8. The nutritional composition of claim 6, wherein the nutritional composition can be administered to further provide partially hydrolyzed guar gum in an amount ranging from about 4 g/day to about 12 grams/day.

9. The nutritional composition of claim 6, wherein the nutritional composition can be administered to further provide partially hydrolyzed guar gum in an amount ranging from about 5 g/day to about 10 grams/day.

10. The nutritional composition of claim 1 comprising at least one ingredient selected from the group consisting of: vitamin, mineral, sugar, a pharmaceutically acceptable carrier, excipient, flavor agent, colorants and combinations thereof.

11. A method of treating a Functional Bowel Disorder in a mammal comprising administering to a mammal in need of same a nutritional composition comprising a therapeutically-effective amount of theanine and an effective amount of exogenous nucleotide.

12. (canceled)

13. The method of claim 11, wherein the Functional Bowel Disorder is selected from the group consisting of: irritable bowel syndrome, Dyspepsia, Functional abdominal pain syndrome, Functional defecation disorders, Functional anorectal disorders, and combinations thereof.

14. The method of claim 13, wherein the Functional anorectal disorders is selected from the group consisting of Functional fecal incontinence, Functional anorectal pain, Chronic proctalgia, Proctalgia fugax, or combinations thereof.

15. The method of claim 11, wherein the nutrition composition is administered to provide theanine in an amount ranging from about 50 mg/day to about 600 grams/day.

16. The method of claim 11, wherein the nutrition composition is administered to provide theanine in an amount ranging from about 100 mg/day to about 400 grams/day.

17. The method of claim 11, wherein the nutrition composition is administered to provide theanine in an amount ranging from about 150 mg/day to about 400 grams/day.

18. The method of claim 11, wherein the nutrition composition is administered to provide theanine in an amount ranging from about 200 mg/day to about 300 grams/day.

19. The method of claim 11, wherein the nutrition composition is administered to provide exogenous nucleotide in an amount ranging from about 200 mg/day to about 6 grams/day.

20. The method of claim 11, wherein the nutrition composition is administered to provide exogenous nucleotide in an amount ranging from about 500 mg/day to about 4 grams/day.

21. The method of claim 11, wherein the nutrition composition is administered to provide exogenous nucleotide in an amount ranging from about 750 mg/day to about 3.5 grams/day.

22. The method of claim 11, wherein the nutrition composition is administered to provide exogenous nucleotide in an amount ranging from about 1 g/day to about 3 grams/day.

23. The method of claim 11, wherein treating is through dietary management.

24. The method of claim 11, wherein the exogenous nucleotide is in a monomeric form selected from the group consisting of 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate and combinations thereof.

25. The method of claim 11, wherein the exogenous nucleotide is intact ribonucleic acid.

26. The method of claim 11, wherein the composition comprises at least one ingredient selected from the group consisting of prebiotic, prebiotic, symbiotic, amino acid, phytonutrients, antioxidant, and combinations thereof.

27. The method of claim 11, wherein the composition comprises partially hydrolyzed guar gum.

28. The method of claim 11, wherein the nutritional composition can be administered and provide partially hydrolyzed guar gum in an amount ranging from about 3 g/day to about 15 grams/day.

29. The method of claim 11, wherein the nutritional composition comprises at least one ingredient selected from the group consisting of: vitamin, mineral, sugar, a pharmaceutically acceptable carrier, excipient, flavor agent, colorants and combinations thereof.
